(19) United States
(12) Patent Application Publication
(10) Pub. No.: US 2026/0021281 A1
(43) Pub. Date: Jan. 22, 2026

(54) SCORING BALLOON CATHETER

(71) Applicant: BOSTON SCIENTIFIC SCIMED, INC., Maple Grove, MN (US)

(72) Inventors: Javier Palomar-Moreno, Galway (IE); Olena Pernatiy, Galway (IE); Anthony Malone, Galway (IE)

(73) Assignee: BOSTON SCIENTIFIC SCIMED, INC., Maple Grove, MN (US)

(21) Appl. No.: 19/270,822

(22) Filed: Jul. 16, 2025

Related U.S. Application Data
(60) Provisional application No. 63/672,875, filed on Jul. 18, 2024.

(51) Int. Cl. A61M 25/10; A61B 17/00; A61B 17/22; A61B 17/32

(52) U.S. Cl. CPC A61M 25/1002; A61B 17/00234; A61B 17/22; A61B 17/32; A61B 2017/00238; A61B 2017/00305; A61B 2017/22065; A61M 2025/105; A61M 2025/1072; A61M 2210/12

(57) ABSTRACT

An example apparatus for treatment of a vessel lesion comprises a catheter shaft; a medical balloon coaxially mounted on the catheter shaft, the medical balloon having a proximal end, a distal end and a body portion between the proximal and the distal end of the medical balloon, the body portion having an exterior surface, where the medical balloon is configured to expand from a deflated condition to an inflated condition, and where the exterior surface, when in its inflated condition, has a varying radial cross-section along a length thereof; and a scoring member having a proximal end, a distal end, and a body portion therebetween, where the body portion of the scoring member is engaged with the exterior surface, and where, when the medical balloon is in the inflated condition, a portion of the scoring member is configured to contact a target site with improved scoring force.

### CROSS REFERENCE TO RELATED APPLICATIONS

The present application claims the benefit of U.S. Provisional Patent Application Ser. No. 63/672,875, filed Jul. 18, 2024, the disclosure of which is incorporated herein by reference.

### TECHNICAL FIELD

The present disclosure pertains to angioplasty and angioplasty medical balloon catheters. More particularly, the present disclosure pertains to angioplasty medical balloon catheters including a medical balloon with a scoring member on exterior surface which has a varying radial cross-section to increase a scoring force of the scoring member. The present disclosure also pertains to drug coated balloons, drug eluting technologies and enhanced drug transfer to a target site (e.g., arterial wall) by inducing therapeutic dissections at the same time as drug transfer takes place.

### BACKGROUND

Arterial blockages, which are also called stenosis, lesions, stenotic lesions, etc., are typically caused by the build-up of atherosclerotic plaque on the inside wall of an artery. In fact, several such stenoses may occur contiguously within a single artery. This can result in a partial, or even complete, blockage of the artery. As a result of the danger associated with such a blockage, several methods and procedures have been developed to treat stenoses. One such method is an angioplasty procedure which uses an inflatable medical balloon to dilate the blocked artery.

Angioplasty medical balloons have enjoyed widespread acceptance in the treatment of stenoses. The efficacy of the dilation of a stenosis may be enhanced by first, or simultaneously, incising the material that is creating the stenosis. Consequently, developments have been made to equip angioplasty medical balloons with cutting edges, or atherotomes, which are intended to incise a stenosis during the dilation procedure. For example, inflatable angioplasty medical cutting balloons having a number of atherotomes mounted longitudinally on the surface of the medical balloon may be employed. Upon inflation of the medical cutting balloon, the atherotomes induce a series of longitudinal cuts into the surface of the stenotic material as the medical balloon expands to dilate the stenosis. As a result of such cuts, the stenosis is more easily dilated, and the likelihood of damaging the artery during dilation is reduced. If a stent is required, the risk of stent under expansion is reduced if these balloons are used for lesion preparation, as arterial plaque is modified and calcified lesions are disrupted.

Blades in many existing cutting medical balloon assemblies tend to be fairly rigid, particularly in the axial direction. The rigid axial structure of the blade naturally limits the blades ability to elongate with the underlying medical balloon material during balloon expansion. Existing blades (e.g., fixed blades) also tend to be fairly rigid in the transverse direction as well. This has the effect of limiting the flexibility of the medical balloon as it is advanced through the tortuous confines of a vessel or other body lumen.

Moreover, existing angioplasty medical balloons (e.g., cutting medical balloons) are often manifested as an individual cylindrical or substantially round medical balloon that has a rounded exterior surface (having a relatively uniform rounded cross-section along a body portion of a medical balloon), when expanded (e.g., when in an inflated condition). As such, a radial cutting force imparted by cutting members (e.g., blades) of the medical balloon may be distributed over a relatively large area (e.g., along an entire length of the round exterior surface and/or along a majority or all of a length of the cutting member). However, lesions may become calcified or otherwise hardened with plaque over time, and thus may present difficulties when attempting to incise the hardened lesions. Thus, the existing cutting medical balloons may be ineffective at providing a sufficient cutting force to incise the hardened lesions. Moreover, physicians may have concerns with tracking or navigating bulky drug coated balloons, such as those with one or more cutting blades through a vessel, particularly from a safety standpoint. For instance, atherotomes or blades, such as those that are several millimeters in length, may have difficulties navigating along vasculatures, such as difficulty navigating bends along a vessel. There is an ongoing need for improved cutting or scoring medical balloons.

### SUMMARY

A first example is an apparatus for treatment of a vessel lesion. The apparatus includes a catheter shaft and a medical balloon coaxially mounted on the catheter shaft. The medical balloon has a proximal end, a distal end and a body portion between the proximal end and the distal end of the medical balloon. The body portion has an exterior surface. The medical balloon is configured to expand from a deflated condition to an inflated condition. The exterior surface, when in its inflated condition, has a varying radial cross-section along a length thereof. A scoring member is provided, having a proximal end, a distal end, and a body portion therebetween. The body portion of the scoring member is engaged with the exterior surface, such that when the medical balloon is in the inflated condition, a portion of the scoring member is configured to contact a target site with improved scoring force.

Alternatively or additionally to any of the examples herein, in another example, the varying radial cross-section is defined by a plurality of peaks and at least one valley therebetween.

Alternatively or additionally to any of the examples herein, in another example, the medical balloon includes at least one annular band configured to form an intermediate waist of the medical balloon which is located between a proximal waist and a distal waist of the medical balloon, or the medical balloon is a molded medical balloon.

Alternatively or additionally to any of the examples herein, in another example, a total quantity of the plurality of peaks is in a range from two peaks to six peaks.

Alternatively or additionally to any of the examples herein, in another example, each peak of the plurality of peaks have the same radial cross-section.

Alternatively or additionally to any of the examples herein, in another example, each peak of the plurality of peaks are equidistantly spaced.

Alternatively or additionally to any of the examples herein, in another example, each peak of the plurality of peaks are the same size, same shape, or both the same size and the same shape.

Alternatively or additionally to any of the examples herein, in another example, the plurality of peaks and the at least one valley together are sinusoidal-shaped.

Alternatively or additionally to any of the examples herein, in another example, the apparatus includes an inflation lumen fluidically coupled to each of the plurality of peaks via one or more inflation ports.

Alternatively or additionally to any of the examples herein, in another example, the scoring member extends along the entire length of the exterior surface.

Alternatively or additionally to any of the examples herein, in another example, the scoring member extends substantially longitudinally in a sinusoidal manner along the entire length of the exterior surface.

Alternatively or additionally to any of the examples herein, in another example, the scoring member is configured to form an annular gap between the portion of the scoring member adjacent to the at least one valley and an exterior surface of the at least one valley when the medical balloon is in the inflated condition.

Alternatively or additionally to any of the examples herein, in another example, the scoring member is a scoring wire.

Alternatively or additionally to any of the examples herein, in another example, the scoring wire is coupled to at least a distal tip of the catheter shaft, and the portion of the scoring wire that contacts the target site is a portion of the scoring wire that is coupled to the plurality of peaks.

Alternatively or additionally to any of the examples herein, in another example, the medical balloon further comprises a drug-eluting sleeve that overlays at least a portion of the body portion of the medical balloon.

Another example is an apparatus for treatment of a vessel lesion. The apparatus includes a catheter shaft having a proximal end portion and a distal end portion, and a medical balloon coaxially mounted on the distal end portion of the catheter shaft. The medical balloon has a proximal end, a distal end, and a body portion between the proximal end and the distal end of the medical balloon. The body portion has an exterior surface. The medical balloon is configured to expand from a deflated condition to an inflated condition. The exterior surface, when in its inflated condition, has a varying radial cross-section along a length thereof. A plurality of scoring wires are provided, each having a proximal end, a distal end, and a body portion there between. The body portion of each scoring wire of the plurality of scoring wires is engaged with the exterior surface, such that when the medical balloon is in the inflated condition, a portion of at least one of the plurality of scoring wires is configured to contact the vessel lesion with improved scoring force.

Alternatively or additionally to any of the examples herein, in another example, each scoring wire of the plurality of scoring wires protrudes radially a distance from the exterior surface.

Alternatively or additionally to any of the examples herein, in another example, the improved scoring force is at least five percent greater than a comparative scoring force associated with a scoring member engaged with an exterior surface having a uniform radial cross-section along a length thereof.

Another example is an apparatus for treatment of a vessel lesion. The apparatus includes a catheter shaft having a proximal end portion, a distal end portion, and an individual inflation lumen extending therethrough. A medical balloon is coaxially mounted on the distal end portion of the catheter shaft. The medical balloon has a proximal end, a distal end, and a body portion between the proximal end and the distal end of the medical balloon. The body portion has an exterior surface. The medical balloon is configured to expand from a deflated condition to an inflated condition. The exterior surface, when inflated via the individual inflation lumen to the inflated condition, has a plurality of peaks and at least one valley therebetween. A pair of diametrically opposed scoring wires are provided, each having a proximal end, a distal end, and a body portion therebetween. The body portion of each of the diametrically opposed scoring wires is engaged with the exterior surface of the medical balloon, such that when the medical balloon is in the inflated condition, the diametrically opposed scoring wires are configured to contact diametrically opposed surfaces of the vessel lesion with improved scoring force.

Alternatively or additionally to any of the examples herein, in another example, the apparatus includes an annular band that is configured to form an intermediate waist that defines the plurality of peaks and the at least one valley.

The above summary of some embodiments, aspects, and/or examples is not intended to describe each disclosed embodiment or every implementation of the present disclosure. The figures and detailed description which follow more particularly exemplify these embodiments.

### BRIEF DESCRIPTION OF THE DRAWINGS

FIG. 1 is a partial longitudinal view of an example catheter including an example of a scoring medical balloon in an inflated condition disposed in a blood vessel;

FIG. 2 is a perspective view of a portion of an example catheter including an example of a scoring wire mounted onto a medical balloon with a plurality of peaks and at least one valley in between, in an inflated condition;

FIG. 3 is a perspective view of a portion of another example catheter including another example of a scoring medical balloon in an inflated condition where a flexible sleeve loaded with therapeutic drug (i.e., anti-proliferative, anti-inflammatory, anti-thrombotic, etc.) has been added;

FIG. 4 is a partial perspective view of a portion of the example catheter of FIG. 3;

FIG. 5 is a perspective exploded view of a portion of the example catheter including a medical balloon in a deflated condition showing the scoring element (wire) and the drug reservoir (a flexible drug-eluting sleeve where the drug is loaded); and

FIG. 6 is an assembled view of the portion of the example catheter of FIG. 5, including the scoring element (e.g., a wire) and the drug reservoir (a drug-eluting sleeve) assembled.

### DETAILED DESCRIPTION

The following description should be read with reference to the drawings wherein like reference numerals indicate like elements throughout the several views. The detailed description and drawings illustrate example embodiments of the claimed invention.

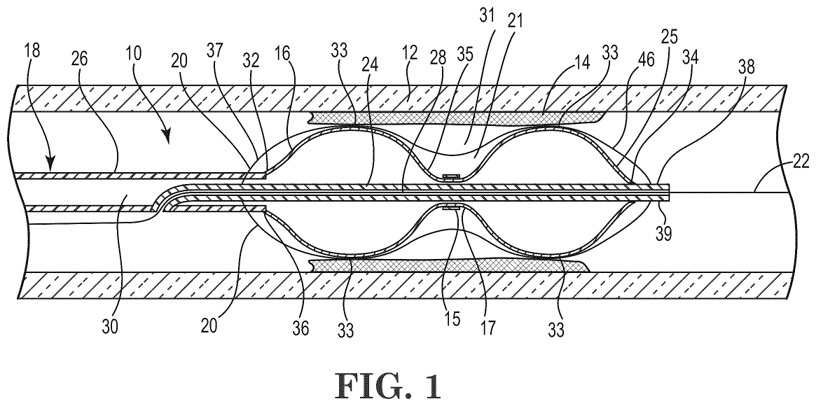

Angioplasty techniques have been shown to be effective for at least some intravascular interventions. FIG. 1 illustrates an example angioplasty catheter 10 positioned in a blood vessel 12 adjacent to a target site 14, namely an intravascular lesion. The target site 14 refers to stenosis or stricture, such as a hardened vessel lesion (e.g., a calcified lesion) or a soft plaque build-up (e.g., cholesterol), within the blood vessel 12. For instance, in some examples the target site 14 refers to hardened vessel lesion (e.g., a calcified lesion).

Catheter 10 (e.g., an apparatus) may include a medical balloon 16 coupled to a catheter shaft 18. A scoring member 20, as detailed herein, may be coupled to catheter shaft 18 and/or medical balloon 16. In general, catheter 10 may be advanced over a guidewire 22 through the vasculature to a target site 14. Medical balloon 16 can then be inflated to cut/score and expand a target site 14 such as a lesion. The target area may be within any suitable peripheral or cardiac location, for example.

In at least some embodiments, the medical balloon 16 may be manufactured from a lubricious material. Alternatively, the medical balloon 16 and catheter 10 may be coated with a lubricious material. Lubricity may be desirable for a number of reasons, such as to enhance the ability of medical balloon 16 to be navigated through the vasculature, particularly when advancing catheter 10 through a relatively narrow or occluded vessel and to minimize friction against ancillary devices such as guide catheters. In these embodiments as well as other embodiments that utilize other medical balloon materials, a preferred embodiment of the present disclosure includes at least one scoring member 20 to provide improved scoring force and/or enhance traction. As used herein, a scoring medical balloon refers to a medical balloon having at least one scoring member 20 disposed on an exterior surface 25 thereof. The at least one scoring member 20 can protrude a distance radially outward from the exterior surface 25 of the medical balloon 16 and thereby permit scoring a lesion, when the medical balloon 16 is in an inflated condition. For instance, each of the scoring members 20 of a plurality of scoring members 20 can protrude the same distance radially outward from a respective portion of the exterior surface 25 (e.g., from a respective peak) of the medical balloon 16 to promote providing a uniform improved scoring forces at each of the portions of the scoring members 20 that contacts a target site.

In general, the medical balloon 16 and the scoring member 20 may each be configured to improve the scoring force imparted by the scoring member 20 on a target site 14 (e.g., lesion) when using catheter 10. For example, scoring member 20 can provide a surface that can help maintain the position of medical balloon 16 (e.g., keep medical balloon 16 from slipping away from target site 14) and/or can provide an improved scoring force, as detailed herein. Therefore, the scoring member 20 can allow for successful scoring or otherwise incising lesions such as hardened lesions (e.g., calcified or otherwise hardened vessel lesions) and scoring lesions having soft plaque (e.g., cholesterol), and yet may also afford greater control when positioning medical balloon 16 and may allow target site 14 (hardened lesion, soft plaque) to be expanded more precisely and prepare the lesion site for stenting, if scaffolding is needed, facilitating correct stent expansion. The medical balloon 16 and the scoring member 20 (e.g., a rounded/circular or triangular scoring element, like a wire) are easier to navigate and at least therefore yield less risk of vessel dissection/perforation, for instance as compared to commercially available cutting blades. Moreover, the medical balloon 16 and the scoring member 20 herein do not require or include the hinge points such as a hinge point that may typically be employed on commercially available cutting blades or atherotomes and which can partially detach the atherotome/blade from the balloon particularly in tortuous vasculature. Additionally, the medical balloon 16 and/or the scoring member 20 herein are shorter in length (a longitudinal length) and/or the medical balloon 16 does not require folding, thus at least for these reasons medical balloon 16 with the scoring member 20 is comparatively easier to navigate (e.g., as the medical balloon 16 can conform to a bend in a vessel) and yet also permit the drug coating to de-bond from the medical balloon 16 and readily transfer to a target site (e.g., vessel wall) when the medical balloon 16 is inflated. The medical balloon 16 and the scoring member 20 also permit drug transfer to a target site (e.g., arterial wall) by inducing therapeutic dissections at the same time as drug transfer takes place. Some of the other features, characteristics, and alternative embodiments of the scoring member 20 are described in more detail below.

Medical balloon 16 can be a compliant, non-compliant, or semi-compliant balloon. For instance, in some embodiments the medical balloon 16 can be a semi-compliant balloon. The medical balloon 16 can be made from typical angioplasty medical balloon materials including polymers such as polyethylene terephthalate (PET), polyetherimide (PEI), polyethylene (PE), etc. Some other examples of suitable polymers, including lubricious polymers, may include polytetrafluoroethylene (PTFE), ethylene tetrafluoroethylene (ETFE), fluorinated ethylene propylene (FEP), polyoxymethylene (POM), polybutylene terephthalate (PBT), polyether block ester, polyurethane, polypropylene (PP), polyvinylchloride (PVC), polyether-ester (for example, a polyether-ester elastomer such as ARNITEL® available from DSM Engineering Plastics), polyester (for example, a polyester elastomer such as HYTREL® available from DuPont), polyamide (for example, DURETHAN® available from Bayer or CRISTAMID® available from Elf Atochem), elastomeric polyamides, block polyamide/ethers, polyether block amide (PEBA, for example, available under the trade name PEBAX®), silicones, MARLEX® high-density polyethylene, MARLEX® low-density polyethylene, linear low density polyethylene (for example, REXELL®), polyetheretherketone (PEEK), polyimide (PI), polyphenylene sulfide (PPS), polyphenylene oxide (PPO), polysulfone, nylon, perfluoro (propyl vinyl ether) (PFA), other suitable materials, or mixtures, combinations, copolymers thereof, polymer/metal composites, and the like. In some embodiments, it may be desirable to use high modulus or generally stiffer materials so as to reduce medical balloon elongation. The above list of materials includes some examples of higher modulus materials. Some other examples of stiffer materials include polymers blended with liquid crystal polymer (LCP) as well as the materials listed above. For example, the mixture can contain up to about 5% LCP. Alternatively, the medical balloon 16 may be coated with a relatively lubricious material such as a hydrogel or silicone.

Shaft 18 may be a catheter shaft, similar to typical catheter shafts which have a distal end portion and a proximal end portion. For example, shaft 18 may include an inner tubular member 24 (e.g., defining a guidewire lumen to accommodate a guide wire and track over it to the target lesion) and outer tubular member 26 (e.g., defining an inflation lumen to allow passage of inflation media, usually contrast dye diluted with saline solution). Tubular members 24/26 may be manufactured from a number of different materials. For example, tubular members 24/26 may be made of metals, metal alloys, polymers, metal-polymer composites or any other suitable materials. Some examples of suitable metals and metal alloys include stainless steel, such as 304V, 304L and 316L stainless steel; nickel-titanium alloy such as linear-elastic or super-elastic Nitinol, nickel-chromium alloy, nickel-chromium-iron alloy, cobalt alloy, tungsten or tungsten alloys, MP35-N (having a composition of about 35% Ni, 35% Co, 20% Cr, 9.75% Mo, a maximum 1% Fe, a maximum 1% Ti, a maximum 0.25% C, a maximum 0.15% Mn, and a maximum 0.15% Si), Hastelloy, Monel 400, Inconel 825, or the like; or other suitable material. Some examples of suitable polymers include those described above in relation to medical balloon 16. Of course, any other suitable polymer may be used without departing from the spirit of the disclosure. The materials used to manufacture inner tubular member 24 may be the same as or be different from the materials used to manufacture outer tubular member 26.

Tubular members 24/26 may be arranged in any appropriate way. For example, in some embodiments inner tubular member 24 can be disposed coaxially within outer tubular member 26. According to these embodiments, inner and outer tubular members 24/26 may or may not be secured to one another along the general longitudinal axis of shaft 18. Alternatively, inner tubular member 24 may follow the inner wall or otherwise be disposed adjacent the inner wall of outer tubular member 26. Again, inner and outer tubular members 24/26 may or may not be secured to one another. For example, inner and outer tubular members 24/26 may be bonded, welded (including tack welding or any other welding technique), or otherwise secured at a bond point. In some embodiments, the bond point may be generally disposed near the distal end portion of shaft 18 or near a proximal end portion of the shaft 18. However, one or more bond points may be disposed at any position along shaft 18. The bond may desirably impact, for example, the stability and the ability of tubular members 24/26 to maintain their position relative to one another. In still other embodiments, inner and outer tubular member 24/26 may be adjacent to and substantially parallel to one another so that they are non-overlapping. In these embodiments, shaft 18 may include an outer sheath that is disposed over tubular members 24/26.

Inner tubular member 24 includes an inner lumen 28. In a preferred embodiment, inner lumen 28 is a guidewire lumen. Accordingly, catheter 10 can be advanced over guidewire 22 to the desired location. The guidewire lumen 28 may be extended along essentially the entire length of catheter shaft 18 so that catheter 10 resembles traditional “over-the-wire” catheters. Alternatively, the guidewire lumen 28 may extend along only a portion of shaft 18 so that catheter 10 resembles “single-operator-exchange” or “rapid-exchange” catheters. Regardless of which type of catheter is contemplated, catheter 10 may be configured so that medical balloon 16 is disposed over at least a region of inner lumen 28. In at least some of these embodiments, inner lumen 28 (i.e., the portion of inner lumen 28 that medical balloon 16 is disposed over) may be substantially coaxial with medical balloon 16.

Shaft 18 may also include an inflation lumen 30 in fluid communication with the interior of the medical balloon 16, that may be used, for example, to transport inflation media to and from medical balloon 16. In some embodiments, the medical balloon 16 can be manifested as a single medical balloon 16. In some embodiments, the inflation lumen 30 can be manifested as a single inflation lumen. In some embodiments, the medical balloon 16 can be a single medical balloon (e.g., including two or more peaks or lobes) and the inflation lumen 30 can be manifested as a single inflation lumen in fluid communication with an interior of each of the lobes or peaks of the medical balloon 16. In such instances, the inflation lumen 30 (a single inflation lumen) can be fluidically coupled to each of the peaks 33 (e.g., lobes) of the medical balloon 16 (a single medical balloon defining multiple peaks 33 or lobes) via one or more inflation ports such that each of the peaks 33 is inflated at the same time with the single inflation lumen 30. For instance, the single inflation lumen 30 can be fluidically coupled to each of the peaks 33 of the medical balloon 16 via a single inflation port at the proximal end of the balloon 16, as illustrated in FIG. 1. Thus, inflation media delivered through the inflation lumen 30 may be introduced into the interior of each of the peaks 33 (e.g., lobes) of the medical balloon 16 in order to inflate the medical balloon 16. However, in some embodiments some but not all of the peaks 33 can be associated with a respective inflation lumen. In other words, in some embodiments, one or more of the peaks 33 can be in fluid communication with a dedicated, separate inflation lumen, in some instances. The balloon 16 can be inflated to a pressure (e.g., about 1600 kilopascals, etc.) in an inflated condition that is typically associated with inflatable medical balloons, for example. Thus, the medical balloon 16 can be employed at typical inflation pressures, and yet can provide improved scoring force, as detailed herein.

The location and position of inflation lumen 30 may vary, depending on the configuration of tubular members 24/26. For example, when outer tubular member 26 is disposed over inner tubular member 24, inflation lumen 30 may be defined within the space between tubular members 24/26. Moreover, depending on the position of inner tubular member 24 within outer tubular member 26, the shape of the inflation lumen 30 (i.e., the shape adjacent shaft 18) may vary and specific tubing, for instance tubing with multiple lumens (e.g., extruded tubing having multiple lumens), can be used to accommodate the guidewire and the inflation media, respectively). For example, if inner tubular member 24 is attached to or disposed adjacent to the inside surface of outer tubular member 26, then inflation lumen 30 may be generally crescent-shaped, for example; whereas if inner tubular member 24 is generally coaxial with outer tubular member 26, then inflation lumen 30 may be generally annular in shape, defining a concentric lumen for the inflation media. It can be appreciated that if outer tubular member 26 is disposed alongside inner tubular member 24, then inflation lumen 30 may be the lumen of outer tubular member 26 or it may be the space defined between the outer surface of tubular members 24/26 and an outer sheath disposed thereover.

In various embodiments, the medical balloon 16 has a varying cross-sectional shape and size along its length, such that two or more peaks 33 or lobes are formed. As used throughout this disclosure, the balloon may include multiple lobes, wherein the radial outermost extent of each lobe defines a peak 33, and the radial innermost extent of the balloon 16 between adjacent lobes defines a valley 35. Stated differently, the varying radial cross-section is defined by a plurality of peaks 33 and at least one valley 35 therebetween. A peak is a portion of the medical balloon which has a greater cross-sectional shape, at least when the medical balloon 16 is in an inflated condition, than portions adjacently proximal and distal thereto. Between the peaks 33, there is a valley 35 in the form of a circumferential trough which has an overall cross-sectional perimeter or shape which is less than that of the peaks. Thus, the balloon 16 may include a valley 35 disposed between adjacent peaks 33 along a length of the balloon 16.

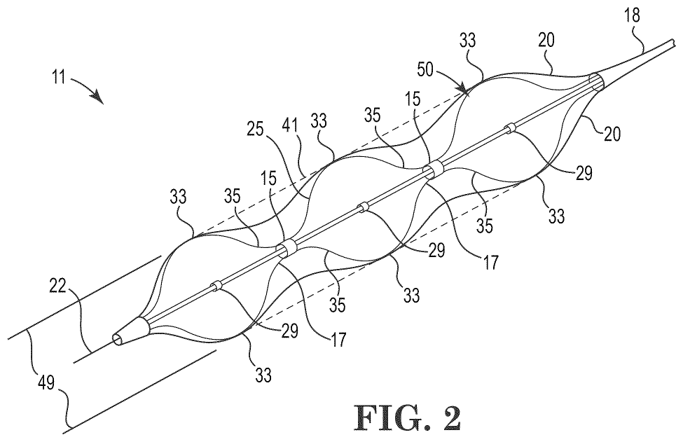

In some embodiments, a total quantity of the peaks 33 can be equal to “N” and a total quantity of the valleys 35 can be equal to “N−1” (e.g., the total quantity of valleys is one less than the total quantity of peaks). Such configurations permit readily treating vascular lesions of different lengths. In some embodiments, a total quantity of peaks can be in a range from two peaks to six peaks or from two peaks to four peaks. For instance, a total quantity of the peaks 33 can be equal to two peaks, three peaks, four peaks, five peaks, or six peaks. In some embodiments, a total quantity of valleys 35 can be in a range from one valley to five valleys or from one valley to three valleys. For instance, a total quantity of the valleys 35 can be one valley, two valleys, three valleys, or four valleys. For example, there can be two peaks and one valley (e.g., as illustrated in FIG. 1) or three peaks and two valleys 35 (e.g., as illustrated in FIG. 2), among other possibilities.

In some embodiments, each peak of the plurality of peaks 33 can be the same size, same shape, or both the same size and the same shape. For instance, in some embodiments each of the peaks of the plurality of peaks 33 can be the same size and the same shape, as illustrated in FIG. 1. For example, in some embodiments each peak of the plurality of peaks 33 can have the same radial cross-section (e.g., cross-section 49 as illustrated in FIG. 2. That is, each of the different peaks can have the same cross-section when taken at the same respective position of the peaks. Employing peaks 33 with the same cross-sections can promote aspects herein such as promoting the portions of the scoring member 20 disposed on the peaks 33 to contact a target site 14 at substantially the same time and/or with substantially the same scoring force (e.g., with an improved scoring force). For instance, each of the peaks 33 can have a radial cross-section in a range from about 2.5 millimeters (0.098 inches) to about 5.7 millimeters (0.224 inches) or from about 2.5 millimeters (0.098 inches) to about 4.5 millimeters (0.177 inches), when the medical balloon 16 is in an inflated condition. Other values of the radial cross-sections of the peaks 33 are possible, and may be varied, for instance, to match the diameters of the different vessels in the vasculature. The radial cross-section of the at least one valley 35 is less than the radial cross-section of the peaks 33, which may define a serrated or sinusoidal longitudinal profile when the balloon is inflated in the treated vessel. For instance, the radial cross-section (taken at an intermediate waist 17 formed by the annular band 15) of the at least one valley 35 can be less than about 3 millimeters or less than about 2 millimeters, among other values, when the balloon 16 is in an inflated condition. While FIG. 1 illustrates each of the peaks 33 having the same radial cross-section, in some embodiments one or more of the plurality of peaks 33 can be a different size and/or different shape than another one of the plurality of peaks 33.

The peaks 33 are configured to engage the scoring member 20 to apply a radially outward force against the scoring member 20 when the balloon 16 is inflated, which in turn exerts a scoring force against the target site 14. In some embodiments, a distance between an apexes of the plurality of peaks 33 can be configured to provide the scoring member 20 with a desired scoring force and/or longitudinal scoring length when the medical balloon 16 is in an inflated condition. For instance, the distance between apexes of the plurality of peaks 33 can be in range from about 3 millimeters (0.118 inches) to about 8 millimeters (0.315 inches), from about 3 millimeters (0.118 inches) to about 5 millimeters (0.197 inches), or from about 4 millimeters (0.157 inches) to about 6 millimeters (0.236 inches), among other possibilities. Without wishing to be bound by theory, it is believed that employing a larger distance (e.g., greater than about 8 millimeters between adjacent peaks, for instance, between apexes of adjacent peaks) may result in difficulties in navigating the apparatuses herein to a target site in a vasculature and/or may reduce the efficiency of scoring a lesion at the target site (e.g., may have too large of a longitudinal spacing to sufficiently incise a lesion along a longitudinal length thereof).

As illustrated in FIG. 1, each peak of the plurality of the plurality of peaks 33 can be equidistantly spaced. For instance, some configurations may include three or more peaks including a first peak, a second peak, and a third peak, where the first peak, the second peak, and the third peak are each equidistantly spaced (spaced apart). That is, a distance between a position on the first peak and a corresponding position on the second peak can be equal to a distance between the corresponding position on the second peak and a position on the third peak. Employing peaks 33 that are equidistant can promote aspects herein such as promoting the portions of the scoring member 20 disposed on the peaks 33 to contact a target site 14 at substantially the same time and/or with substantially the same scoring force (e.g., with an improved scoring force).

In some embodiments, the plurality of peaks 33 and the at least one valley 35 can together be sinusoidal-shaped. That is, the plurality of peaks 33 and the at least one valley 35 can together form a diamond, serrated, sinusoidal, pearl, or undulating shape, for instance, including two or more conical or diamond shaped regions. For instance, the plurality of peaks 33 and the at least one valley 35 can form two successive substantially bulbous or diamond shaped regions along a longitudinal axis of the catheter 10, as illustrated in FIG. 1. Employing the sinusoidal-shaped configuration of the plurality of peaks 33 and the at least one valley 35 can promote aspects herein such as promoting the navigation of long diffuse lesions and promoting enhanced scoring force by the scoring member 20 when the medical balloon 16 is in an inflated condition. However, other shapes of peaks 33 and/or the at least one valley 35 are possible. For instance, in some embodiments the plurality of peaks 33 and the at least one valley 35 are substantially oval shaped (e.g., pearl-shaped). Employing an oval-shaped configuration of the plurality of peaks 33 and the at least one valley 35 can promote aspects herein such as promoting enhanced scoring force by the scoring member 20 when the medical balloon 16 is in an inflated condition and/or promoting case of delivery to and/or removal of the medical balloon 16 to the target site 14.

The medical balloon 16 can include at least one intermediate waist 17 of the medical balloon 16 defining the valley 35 between adjacent peaks 33. In some embodiments, the medical balloon 16 can include at least one annular band 15 that is configured to form at least one intermediate waist 17 of the medical balloon 16. The intermediate waist 17 is formed along the exterior surface 25 of the medical balloon 16 at a location that is between a proximal waist 32 (e.g., located at or near a proximal end region 37 of the medical balloon 16, as illustrated in FIG. 1) and a distal waist 34 (e.g., located at or near a distal end region 46 of the medical balloon 16, as illustrated in FIG. 1) of the medical balloon 16. The proximal waist 32 may be secured or bonded to the elongate shaft 18 at a proximal end of the balloon 16 and the distal waist 34 may be secured or bonded to the elongate shaft 18 at a distal end of the balloon 16. The intermediate waist(s) 17 may be necked down portions of the balloon 16 which may or may not be secured or bonded to the elongate shaft 18. Thus, an intermediate waist 17 can be configured to define a valley 35 between adjacent peaks 33 or lobes of the balloon 16. In some instances, the medical balloon 16 can include at least one annular band 15 circumferentially surrounding the intermediate waist 17 which constrains a portion of the exterior surface 25 of the medical balloon 16 to form a bottom of a valley 35 from which the remainder of the valley 35 and the adjacent peaks 33 can extend. For instance, in some embodiments the medical balloon 16 can include an annular band 15 at each intermediate waist 17 of the medical balloon 16. The annular band 15 can be formed of an elastic or inelastic material that is configured to constrain radial expansion of a portion of the exterior surface 25 of the medical balloon 16 (e.g., the portions of the medical balloons disposed beneath and in contact with the annular band 15), thereby promoting formation of the at least one valley 35 along with the peaks 33 adjacent thereto.

An inflation passage may extend through the intermediate waist 17, connecting the interior inflation chamber of the peak 33 (e.g., lobe) on a proximal side of the intermediate waist 17 to the interior inflation chamber of the peak 33 (e.g., lobe) on a distal side of the intermediate waist 17. For example, an interior surface of the balloon 16 may be spaced radially outward of the portion of the elongate shaft 18 extending through the intermediate waist 17, providing a space or gap therebetween to act as the inflation passage. Thus, inflation media may flow between the interior inflation chambers of adjacent peaks 33 to inflate the plurality of peaks via a single inflation lumen and inflation media.

In some embodiments, the annular bands 15 can be or include a radiopaque marker coupled thereto that may aid a user in determining the location of catheter 10 within the vasculature. Similarly, in some embodiments, the peaks 33 and/or the at least one valley 35 can include a radiopaque marker coupled thereto, or coupled to an underlying portion of the elongate shaft 18 longitudinally aligned therewith as shown as radiopaque bands 29, that may aid a user in determining the location of catheter 10 (e.g., peaks of the balloon) within the vasculature. While the medical balloon 16 is illustrated as including an annular band 15, other configurations are possible. For instance, in some embodiments the medical balloon 16 can be a molded medical balloon 16 such as a molded medical balloon having a plurality of lobes that has an absence of any annular bands. The molded medical balloon can be formed with the same or substantially similar shape as the medical balloon 16 illustrated in FIG. 1. For example, the molded medical balloon can be formed to include a valley and a plurality of peaks e.g., together forming diamond, serrated, sinusoidal, diamond, or pearl shaped regions, among other possible configurations. The molded medical balloon can be formed by casting and/or via extrusion (e.g., extruded from tubing and/or a die) or other methodologies. For example, the medical balloon can be formed by extrusion followed by balloon molding (e.g., of an extruded tube of material), for instance, using a succession of two conical sections (proximal and distal) and a short cylindrical section (middle). Such molding can be repeated multiple times, for instance, to mold the extruded tube of material into a plurality of peaks and at least one valley therebetween.

One or more scoring members are engaged (e.g., secured or affixed) to at least the distal tip of the catheter 10 and extend proximally therefrom along an exterior of the medical balloon 16. The scoring members 20 may contact or otherwise engage an exterior surface of the medical balloon 16 at the peaks 33, in some instances, the scoring members 20 may not contact or otherwise engage the exterior surface of the medical balloon between adjacent peaks 33. In other words, the scoring members 20 may span over the valleys 35 without contacting or otherwise engaging the exterior surface of the medial balloon along the valleys 35. Additionally, in some embodiments, the one or more scoring members can be affixed or bonded to the peaks 33 via an adhesive or otherwise engaged to the peaks 33. For instance, the scoring wire can be coupled to at least apexes of the peaks 33 and the distal tip 39 of the catheter shaft 18, as illustrated in FIG. 1. In some embodiments, the scoring member 20 can be engaged to the apexes of the peaks 33 and can be spaced a distance away from the valleys 35. For example, the scoring member 20 may be engaged with each of the peaks 33 and may extend over (but not be engaged with) an expanse formed by the at least one valley 35, at least when the medical balloon 16 is in an inflated condition, as illustrated in FIG. 1. For instance, the scoring member 20 can be a flexible scoring wire that is coupled to the apexes of the peaks 33 and is configured to droop (e.g., is radially closer to the longitudinal axis of the catheter 10) between the peaks 33 such that a portion of the scoring wire between the peaks 33 is located radially inward of an axis 41 (e.g., as illustrated in FIG. 2) or plane extending between adjacent peaks axially aligned with an outermost extent of the peaks 33 such that the outermost extent of adjacent peaks 33 lies on the axis 41, at least when the medical balloon 16 is in an inflated condition. In such embodiments, the portion of the scoring member 20 (wire) that droops radially inward of the axis 41 may remain spaced a distance away from the exterior surface 25 of the at least one valley 35). Thus, the scoring member 20 can be configured to form an annular gap 21 between a portion of the scoring member 20 adjacent to the exterior surface 25 in the valley 35 and the exterior surface 25 in the valley 35 when the medical balloon 16 is in the expanded form. Similarly, an annular gap 31 can be formed between the portion of the scoring member 20 adjacent to the exterior surface 25 of the valley 35 and the target site 14 when the medical balloon 16 is in the expanded form. Providing the annular gap 21 and the annular gap 31 can promote aspects herein such as prompting an improved scoring force by the portions (e.g., the portions of the scoring member 20 that are engaged with the peaks 33) of the scoring member 20 that are in contact with a vasculature (e.g., stenosis or stricture), while one or more other portions of a scoring member 20 (e.g., the portion in the valley 35) are not in contact with the vasculature (e.g., stenosis or stricture).

For instance, unlike some previous approaches which employ scoring balloons with a scoring member on an exterior surface 25 which has a uniform radial cross-section (e.g., those which employ an individual substantially rounded or cylindrical medical balloon) when in an inflated condition), the medical balloon 16 herein has a varying radial cross-section along a longitudinal length of the body portion of the medical balloon 16, as detailed herein. Thus, the medical balloon 16 with the varying radial cross-section along a body portion thereof can reduce an amount of surface area of the exterior surface 25 of the medical balloon 16 that is in contact with a target site 14 (lesion), and thus can provide improved (higher) scoring force at an interface between portions of the scoring member 20 and the target site 14, as detailed herein. For instance, in some embodiments the improved scoring force is at least five percent greater, at least ten percent greater, at least fifteen percent greater, or at least twenty percent greater than a comparative scoring force associated with the scoring member engaged with an exterior surface 25 having a uniform radial cross-section along a length thereof. Thus, employing the apparatuses herein with improved scoring force can ensure that even hardened lesions (e.g., calcified lesions) are cut/disrupted and thereby ensure that patency of a lumen in which the hardened lesion is disposed is improved or restored, in contrast to other approaches such as those which employ scoring balloons with a substantially uniform radial cross-section (e.g., cylindrical scoring balloons) along the body portion thereof.

While FIG. 1 illustrates the medical balloon in an inflated condition, in some embodiments the medical balloon 16 may have the same or similar shape and configuration (albeit with a different size) when the medical balloon 16 is in its nominal or deflated condition.

The scoring member 20 can extend along the entire length of the exterior surface 25 of the body portion of the medical balloon 16. For instance, the scoring member 20 can extend substantially longitudinally (e.g., in a sinusoidal manner) along the entire length of the exterior surface 25 from a location distal of the body portion of the medical balloon 16 to a location proximal of the body portion of the medical balloon 16, as illustrated in FIG. 1. The scoring member 20 can be a flexible scoring member. For instance, the scoring member 20 can be a scoring wire. The scoring wire can have a diameter in a range from about 0.2032 millimeters (0.008 inches) to about 0.3556 millimeters (0.014 inches). In some embodiments, the scoring wire can be a round elongate scoring wire having a substantially circular cross-section; however, other shapes such as those described herein are possible.

In some embodiments, the apparatuses herein can include a pair of diametrically opposed scoring members (e.g., scoring wires) each having a proximal end, a distal end, and a body portion therebetween, where the body portion is engaged with the exterior surface 25 at the peaks 33, and where, when the medical balloon is in the inflated condition, the diametrically opposed scoring members are configured to contact diametrically opposed surfaces of the vessel lesion with improved scoring force (e.g., at the same time). For instance, FIGS. 1-6 each illustrate apparatuses including a pair of diametrically opposed scoring wires (e.g., scoring members 20 as illustrated in FIG. 1 which are located on opposing points of the exterior surface 25 engaging the balloon 16 at the peaks 33 and spaced away from the balloon 16 at the valleys 35). Although the foregoing figures illustrate various example catheters having two scoring members, the disclosure is not intended to be limited to any particular number of scoring members. For instance, in some embodiments the catheters can include an individual scoring member or can include three or more scoring members.

Medical balloon 16 may be coupled to catheter shaft 18 in any of a number of suitable ways. For example, medical balloon 16 may be adhesively or thermally bonded to shaft 18. In some embodiments, the proximal waist 32 of medical balloon 16 may be bonded to shaft 18, for example, at outer tubular member 26, and the distal waist 34 may be bonded to shaft 18, for example, at inner tubular member 24. The exact bonding positions, however, may vary. It can be appreciated that a section of proximal waist 32 may not have section 36 extending therefrom in order for suitable bonding between medical balloon 16 and outer tubular member 26.

In addition to some of the structures described above, shaft 18 may also include a number of other structural elements, including those typically associated with catheter shafts. For example, shaft 18 may include a radiopaque marker coupled thereto that may aid a user in determining the location of catheter 10, such as the lobes or peaks 33 of the balloon 16 within the vasculature. In addition, catheter 10 may include a folding spring (not shown) coupled to medical balloon 16, for example, adjacent proximal waist 32, which may further help in medical balloon folding and refolding. A description of a suitable folding spring can be found in U.S. Pat. Nos. 6,425,882 and 6,623,451, the disclosures of which are incorporated herein by reference.

The attachment point and/or attachment configuration of scoring member 20 may vary. For example, scoring member 20 can be attached to shaft 18 (e.g., at least at the distal tip of the shaft). In at least some of these embodiments, the distal end 38 of scoring member 20 may be attached to shaft 18 at a position distal of medical balloon 16, and a proximal end of scoring member 20 may be attached to shaft 18 at a position proximal of the medical balloon 16. However, this configuration is not intended to be limiting as it is contemplated that any part of scoring member 20 may be attached to shaft 18 at any suitable position. The type of attachment may also vary. For example, scoring member 20 may be attached to shaft 18 by welding, laser bonding, soldering, brazing, adhesive bonding, by using a mechanical fitting or connector, by winding or wrapping scoring member 20 around shaft 18, and the like, or in any other suitable way.

In some embodiments, a drug-eluting sleeve (not shown in FIG. 1) or therapeutic (e.g., drug-eluting) coating may overlay at least a portion of the body of the medical balloon 16, as detailed herein. For instance, a drug-eluting sleeve may be coupled to catheter shaft 18 in any of a number of suitable ways. For example, drug-eluting sleeve may be adhesively or thermally bonded to shaft 18, for instance, at locations along the catheter shaft 18 that are proximal and distal to the medical balloon 16. The drug-eluting sleeve may be configured (e.g., with an appropriate radial thickness and/or with an appropriate excipient) to permit drug-delivery prior to, during, and/or subsequent to scoring of a lesion with the scoring members herein. For instance, the drug-eluting sleeve can be configured to permit drug-delivery to a target site at least while scoring the target site with the scoring member, when the medical balloon is in an inflated condition.

In some embodiments, the drug-eluting sleeve or therapeutic coating on the surface of the balloon can include an excipient, an active agent and/or drug (e.g., an amorphous form of a drug or a crystalline form of a drug). Some specific beneficial agents include anti-thrombotic agents, anti-proliferative agents, anti-inflammatory agents, anti-migratory agents, pro-endothelization agents and/or other agents affecting extracellular matrix production and organization, antineoplastic agents, anti-mitotic agents, anesthetic agents, anti-coagulants, vascular cell growth promoters, vascular cell growth inhibitors, cholesterol-lowering agents, vasodilating agents, and agents that interfere with endogenous vasoactive mechanisms.

More specific drugs or therapeutic agents include paclitaxel, rapamycin, sirolimus, everolimus, tacrolimus, heparin, diclofenac, aspirin, Epo D, dexamethasone, estradiol, halofuginone, cilostazol, geldanamycin, ABT-578 (Abbott Laboratories), trapidil, liprostin, Actinomycin D, Resten-NG, Ap-17, abciximab, clopidogrel, Ridogrel, beta-blockers, bARKct inhibitors, phospholamban inhibitors, and SERCA 2 gene/protein, resiquimod, imiquimod (as well as other imidazoquinoline immune response modifiers), human apolipoproteins (e.g., AI, AII, AIII, AIV, AV, etc.), vascular endothelial growth factors (e.g., VEGF-2), as well as derivatives of the forgoing, among many others. In some embodiments, the drug may be a macrolide immunosuppressive (limus) drug. Other drugs may include anti-inflammatory agents such as dexamethasone, prednisolone, corticosterone, budesonide, estrogen, sulfasalazine, mesalamine, and analogues thereof; antineoplastic/antiproliferative/anti-miotic agents such as paclitaxel, 5-fluorouracil, cisplatin, vinblastine, vincristine, epothilones, endostatin, angiostatin, thymidine kinase inhibitors, and analogues thereof; anesthetic agents such as lidocaine, bupivacaine, ropivacaine, and analogues thereof; anti-coagulants; and growth factors.

In some instances, the drug-eluting sleeve or therapeutic coating may include individual drug particles that are encapsulated with one or more excipients. The drug particles may include crystals of the drug, for example. Drug crystals may be formed in a variety of ways, for example. In some cases, a drug or other therapeutic agent may be available in an amorphous form, and a variety of processes may be used to convert an amorphous drug or other therapeutic agent into a crystalline drug or other therapeutic agent.

In some instances, a drug-eluting sleeve can be disposed on (overlay and be in contact with) the peaks and proximal and distal waists of a medical balloon of the medical apparatuses (e.g., medical devices) herein. For example, a drug-eluting sleeve can be slid over a medical balloon when the medical balloon is in a deflated condition and can be bonded (e.g., via thermal bonding or adhesive bonding to portions of the medical balloon such as a proximal waist and a distal waist of the medical balloon. However, in some instances, the medical device may be contacted with the coating composition in order to form a coating on the medical device. In some instances, the medical device or a portion thereof may be dipped into the coating composition. In some cases, vapor deposition may be used to transfer the coating composition to the medical device. In some cases, a roller coating process may be used to transfer the coating composition/formulation to the medical device. In some cases, ink-jet printing technology may be used to apply the coating composition/formulation to the medical device. These are just examples. In some cases, the coating composition may be sprayed onto the medical device, or may be sprayed onto a particular portion or region of the medical device.

In some instances, one or more excipients may be employed. For instance, a mixture of excipients may be employed that includes two or more different excipients. An example excipient may include ethyl cellulose (EC), which is a derivative of cellulose in which some of the hydroxyl groups on the repeating glucose units are converted into ethyl ether groups. The relative number of ethyl ether groups can vary depending on the particular manufacturer. EC has the following chemical structure:

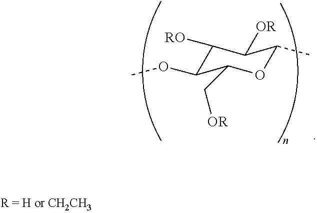

Another example excipient may include acetyl tri-butyl citrate (ATBC), which in some cases may be referred to by its IUPAC name of tributyl 2-acetyloxypropane-1,2,3-tricarboxylate. ATBC has the following chemical structure:

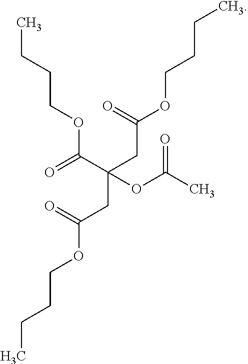 [SMILES: CCCCOC(=O)CC(CC(=O)OCCCC)(OC(C)=O)C(=O)OCCCC]

In some cases, the mixture of excipients may optionally include one or more additional excipients, to ensure that the active pharmaceutical ingredient (drug) is retained by the coated sleeve while the catheter navigates the vasculature and transferred to the lesion upon balloon inflation (which stresses the elastic sleeve, releasing the drug).

FIGS. 2-6 illustrate alternative example embodiments of catheters that include various example scoring medical balloons. For instance, FIG. 2 is a perspective view of a portion of an example catheter 11 including an example of a medical balloon 50 in an inflated condition. The example catheter 11 of FIG. 2 is analogous to the example catheter 10 of FIG. 1, with the change that the catheter 11 includes medical balloon 50 which has a total of three peaks 33 and two valleys 35 therebetween (as compared to the two peaks 33 and the single valley 35 of the medical balloon 16). Employing additional peaks (e.g., three peaks) can promote aspects herein such as ensuring a sufficient scoring length (e.g., extending from a first proximal peak to a third distal peak) to cut along a majority or an entirety of a vessel lesion such as a hardened lesion or soft plaque built-up of greater length, or a diffuse calcified lesion. That is, as mentioned previously the approaches herein can yield scoring medical balloons that can be tailored (e.g., based on a radial cross-section and/or a quantity of peaks) to cut different sized soft plaque and hardened lesions (e.g., having differing longitudinal lengths in a vasculature and/or being disposed at targets sites within vessels of different diameters).

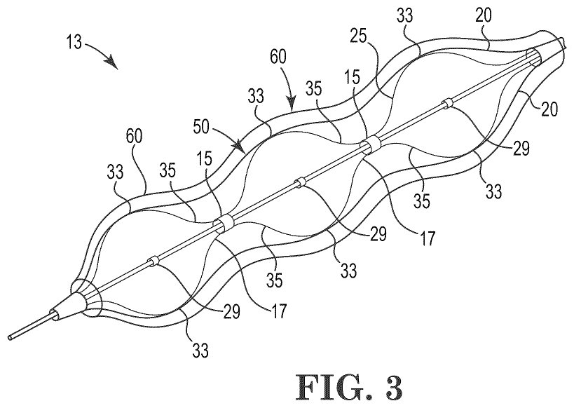

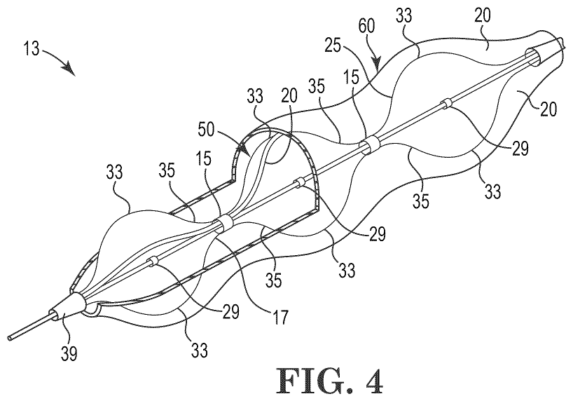

FIG. 3 is a perspective view of a portion of another example catheter 13 including another example of a scoring medical balloon 50 in an inflated condition. The example catheter 13 of FIG. 3 is analogous to the catheter 11 of FIG. 2, with the change that the catheter 13 includes a drug-eluting sleeve 60 disposed on the medical balloon 50. As illustrated in FIG. 3, the drug-eluting sleeve 60 can extend circumferentially around an entire exterior surface 25 of the medical balloon 50 (e.g., circumferentially around at least each of the peaks 33 of the medical balloon 50. For instance, FIG. 4 is a partial perspective view of a portion of the example catheter 13 of FIG. 3.

As illustrated in FIG. 4, the drug-eluting sleeve 60 can extend circumferentially around and overlay each of the components of the medical balloon 50. For instance, the drug-eluting sleeve 60 can extend circumferentially around and overlay the scoring members 20 and at least the peaks 33 of the medical balloon 50. In some instances, the drug-eluting sleeve 60 may have a longitudinal length at least as long as the distance between the proximalmost peak 33 and the distalmost peak 33 of the medical balloon 50. In such instances, the scoring members 20 may radially protrude into and/or through the drug-eluting sleeve 60, or otherwise exert a radial force on a portion of the drug-eluting sleeve to contact (directly contact) a target site (e.g., a lesion), when the medical balloon 50 is in an expanded condition, among other possibilities.

As mentioned previously, the presence of the drug-eluting sleeve 60 can permit drug delivery prior to, during, and/or after scoring a lesion (e.g., a hardened lesion) with the scoring members 20. For instance, the example catheter 13 can be configured to provide drug-delivery via the drug-eluting sleeve 60 to a target site at the same time as the scoring members 20 score the target site (e.g., a hardened lesion). Providing drug-delivery to the target site at least at the same time as the scoring members 20 score the target site can promote aspects herein such as mitigating inflammation associated with scoring the target site, thereby reducing a likelihood of the target site forming a new lesion and/or otherwise mitigating any unintended side-effects associated with scoring the target site. The scoring can induce therapeutic dissections on the vessel wall to enhance drug transfer and drug retention in the treated vessel wall.

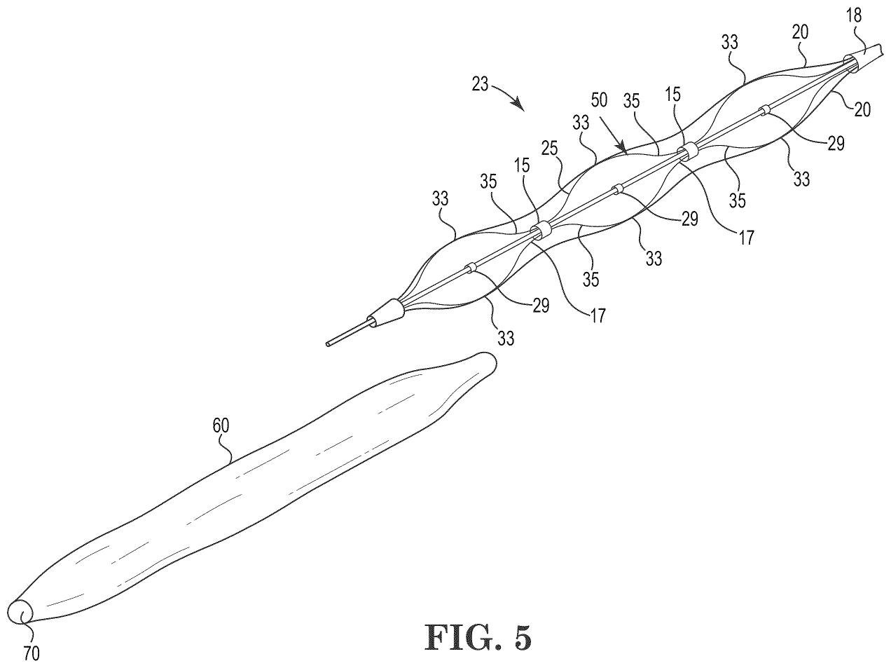

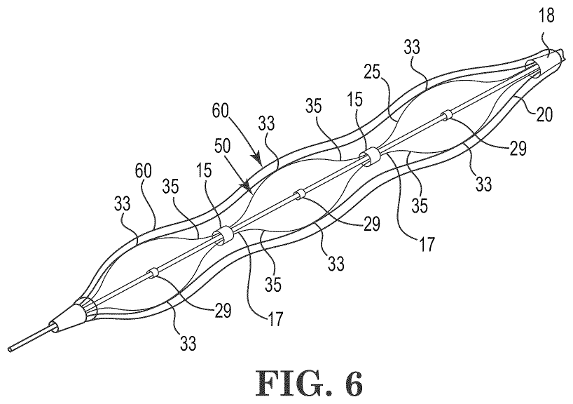

FIG. 5 is a perspective exploded view of a portion of the example catheter 23 including medical balloon 50 of FIG. 6 in a deflated condition. As illustrated in FIG. 5, the drug-eluting sleeve 60 can be a separate component from the medical balloon 50 which allows for chemistry and manufacturing controls during manufacture, as the drug-eluting sleeve 60 can be weighed before and after applying the drug load thereto. The drug-eluting sleeve 60 can be tested analytically to verify correct dosage and it can be assembled over the medical balloon 50 affixing it by means of adhesive, thermal bonding, laser bonding in one or more locations (i.e., proximal and distal to the plurality of peaks 33). For instance, the drug-eluting sleeve 60 can be an elastic tubular member having a lumen 70 extending substantially longitudinally therethrough. The lumen 70 can be configured (e.g., shaped and sized) to receive at least a portion of the medical balloon 50. For instance, an entirety of the medical balloon 50 or at least the body portion including the peaks 33 of the medical balloon 50 can be disposed within the lumen 70. For example, the medical balloon 50 can be disposed within the lumen 70 when the medical balloon 50 is in a deflated condition and subsequently one or more ends of the drug-eluting sleeve 60 can be affixed to the catheter shaft 18 and/or the medical balloon 50, as detailed herein. For instance, FIG. 6 is an assembled view of a portion of the example catheter 23 including the medical balloon 50 of FIG. 5 in a deflated condition, in which the proximal and distal ends of the drug-eluting sleeve 60 are each affixed to respective portions of the catheter shaft 18. Namely, the proximal end of the drug-eluting sleeve 60 may be affixed or bonded to the catheter shaft 18 proximal of the medical balloon 50, and the distal end of the drug-eluting sleeve 60 may be affixed or bonded to the catheter shaft 18 distal of the medical balloon 50, such that the drug-eluting sleeve 60 fully surrounds the medical balloon 50 and the scoring members 20 therebetween.

In some embodiments, the location (e.g., a proximal attachment point and/or a distal attachment point) of a scoring member and/or a medical balloon can be at different locations than those depicted herein. For instance, in some embodiments the distal end of the scoring member can be attached to the medical balloon and/or the catheter shaft, while the proximal end of the scoring member is allowed to float (e.g., within an outer tube and/or an inner tube of the catheter shaft). Permitting the proximal end of the scoring member to float (e.g., remain unattached to a fixed point) can impart an added degree of flexibility in the apparatuses herein, for instance, to permit the scoring member to bend as may be needed during delivery, deployment, and/or responsive to a change in a condition (e.g., in an inflated condition, a deflated condition, etc.) of the medical balloon.

The representative embodiments herein show the scoring members 20 as substantially round wires (having a circular cross-section) extending substantially longitudinally in an undulating (e.g., sinusoidal) manner. It is, however, contemplated that any or all of the scoring members may extend over any portion of the medical balloon in an alternative direction. For example, a scoring member may extend radially around the medical balloon. Alternatively, a scoring member may follow a generally helical pattern over the medical balloon.

Moreover, embodiments that include a plurality of scoring members may include a plurality of the same or similar scoring members or combination of differing scoring member embodiments (e.g., with differing thicknesses, differing shapes, and/or differing orientations, etc.), including any of those described herein. In some embodiments, the scoring members 20 may take a different shape such as a triangular shape where an apex of the triangle is configured to extend radially from the exterior surface (e.g., exterior surface 25 as illustrated in FIG. 1). In such instances, the triangular shaped scoring wire can further reduce an amount of surface area of the scoring member (e.g., the apex of the triangle) in contact with a target site and thereby can provide a further improvement in an amount of scoring force imparted by the scoring wire on the target site. In such embodiments, a base side of the triangle can be disposed (contact) the exterior surface of the medical balloon. Of course, other shapes can be used in various forms of scoring members without departing from the spirit of the disclosure. For example, various embodiments of scoring members may have circular, square, rectangular, polygonal, or any other suitable cross-sectional shape. The degree of curvature, pattern of curvature, and positioning of curves along the length of scoring members may also vary to include essentially any appropriate configuration.

In at least some embodiments, portions or all of the medical apparatuses (devices) described herein may also be doped with, made of, or otherwise include a radiopaque material. Radiopaque materials are understood to be materials capable of producing a relatively bright image on a fluoroscopy screen or another imaging technique during a medical procedure. This relatively bright image aids the user of the medical devices described herein in determining its location. Some examples of radiopaque materials can include, but are not limited to, gold, platinum, palladium, tantalum, tungsten alloy, polymer material loaded with a radiopaque filler, and the like. Additionally, other radiopaque marker bands and/or coils may also be incorporated into the design of the medical devices described herein to achieve the same result.

In some embodiments, a degree of Magnetic Resonance Imaging (MRI) compatibility is imparted into the medical apparatuses described herein. For example, the medical apparatuses described herein, or portions thereof, may be made of a material that does not substantially distort the image and create substantial artifacts (i.e., gaps in the image). Certain ferromagnetic materials, for example, may not be suitable because they may create artifacts in an MRI image. The medical devices described herein, or portions thereof, may also be made from a material that the MRI machine can image. Some materials that exhibit these characteristics include, for example, tungsten, cobalt-chromium-molybdenum alloys (e.g., UNS: R30003 such as ELGILOY®, PHYNOX®, and the like), nickel-cobalt-chromium-molybdenum alloys (e.g., UNS: R30035 such as MP35-N® and the like), nitinol, and the like, and others.

It should be understood that this disclosure is, in many respects, only illustrative. Changes may be made to various details herein, particularly in matters of shape, size, and arrangement of steps without exceeding the scope of the disclosure. This may include, to the extent that it is appropriate, the use of any of the features of one example embodiment being used in other embodiments. The scope of the disclosure is, of course, defined in the language in which the appended claims are expressed.

What is claimed is:

1. An apparatus for treatment of a vessel lesion, the apparatus comprising:
a catheter shaft;
a medical balloon coaxially mounted on the catheter shaft, the medical balloon having a proximal end, a distal end and a body portion between the proximal end and the distal end of the medical balloon, said body portion having an exterior surface, wherein the medical balloon is configured to expand from a deflated condition to an inflated condition, and wherein the exterior surface, when in its inflated condition, has a varying radial cross-section along a length thereof; and
a scoring member having a proximal end, a distal end, and a body portion therebetween, wherein the body portion of the scoring member is engaged with the exterior surface, and wherein, when the medical balloon is in the inflated condition, a portion of the scoring member is configured to contact a target site with improved scoring force.

2. The apparatus of claim 1, wherein the varying radial cross-section is defined by a plurality of peaks and at least one valley therebetween.

3. The apparatus of claim 2, wherein:
the medical balloon includes at least one annular band configured to form an intermediate waist of the medical balloon which is located between a proximal waist and a distal waist of the medical balloon; or
the medical balloon is a molded medical balloon.

4. The apparatus of claim 2, wherein a total quantity of the plurality of peaks is in a range from two peaks to six peaks.

5. The apparatus of claim 2, wherein each peak of the plurality of peaks have the same radial cross-section.

6. The apparatus of claim 2, wherein each peak of the plurality of peaks are equidistantly spaced.

7. The apparatus of claim 2, wherein each peak of the plurality of peaks are the same size, same shape, or both the same size and the same shape.

8. The apparatus of claim 2, wherein the plurality of peaks and the at least one valley together are sinusoidal-shaped.

9. The apparatus of claim 2, wherein the apparatus includes an inflation lumen fluidically coupled to each of the plurality of peaks via one or more inflation ports.

10. The apparatus of claim 1, wherein the scoring member extends along the entire length of the exterior surface.

11. The apparatus of claim 10, wherein the scoring member extends substantially longitudinally in a sinusoidal manner along the entire length of the exterior surface.

12. The apparatus of claim 1, wherein the scoring member is configured to form an annular gap between a portion of the scoring member adjacent to the at least one valley and an exterior surface of the at least one valley when the medical balloon is in the inflated condition.

13. The apparatus of claim 1, wherein the scoring member is a scoring wire.

14. The apparatus of claim 13, wherein:
the scoring wire is coupled to at least a distal tip of the catheter shaft; and
the portion of the scoring wire that contacts the target site is a portion of the scoring wire that is coupled to the plurality of peaks.

15. The apparatus of claim 1, wherein the medical balloon further comprises a drug-eluting sleeve that overlays at least a portion of the body portion of the medical balloon.

16. An apparatus for treatment of a vessel lesion, the apparatus comprising:
a catheter shaft having a proximal end portion and a distal end portion;
a medical balloon coaxially mounted on the distal end portion of the catheter shaft, the medical balloon having a proximal end, a distal end and a body portion between the proximal end and the distal end of the medical balloon, the body portion having an exterior surface, wherein the medical balloon is configured to expand from a deflated condition to an inflated condition, and wherein the exterior surface, when in its inflated condition, has a varying radial cross-section along a length thereof; and
a plurality of scoring wires each having a proximal end, a distal end, and a body portion therebetween, wherein the body portion of each scoring wire of the plurality of scoring wires is engaged with the exterior surface, and wherein, when the medical balloon is in the inflated condition, a portion of at least one of the plurality of scoring wires is configured to contact the vessel lesion with improved scoring force.

17. The apparatus of claim 16, wherein each scoring wire of the plurality of scoring wires protrudes radially a distance from the exterior surface.

18. The apparatus of claim 17, wherein the improved scoring force is at least five percent greater than a comparative scoring force associated with a scoring member engaged with an exterior surface having a uniform radial cross-section along a length thereof.

19. An apparatus for treatment of a vessel lesion, the apparatus comprising:
a catheter shaft having a proximal end portion and a distal end portion and including an individual inflation lumen;
a medical balloon coaxially mounted on the distal end portion of the catheter shaft, the medical balloon having a proximal end, a distal end and a body portion between the proximal end and the distal end of the medical balloon, the body portion having an exterior surface, wherein the medical balloon is configured to expand from a deflated condition to an inflated condition, and wherein the exterior surface, when inflated via the individual inflation lumen to the inflated condition, has a plurality of peaks and at least one valley therebetween; and
a pair of diametrically opposed scoring wires each having a proximal end, a distal end, and a body portion therebetween, wherein the body portion of each of the diametrically opposed scoring wires is engaged with the exterior surface, and wherein, when the medical balloon is in the inflated condition, the diametrically opposed scoring wires are configured to contact diametrically opposed surfaces of the vessel lesion with improved scoring force.

20. The apparatus of claim 19, including an annular band that is configured to form an intermediate waist that defines the plurality of peaks and the at least one valley.
